(19) United States
(12) Patent Application Publication
(10) Pub. No.: US 2011/0015476 A1
(43) Pub. Date: Jan. 20, 2011

(54) Devices and Methods for Treating Cardiomyopathy

(76) Inventor: Jeff Franco, Clarksville, MD (US)

Correspondence Address: JUNEAU PARTNERS, 333 N Fairfax St., Suite 305, ALEXANDRIA, VA, 22301, US

(21) Appl. No.: 12/589,544

(22) Filed: Oct. 14, 2009

Related U.S. Application Data
(60) Provisional application No. 61/157,209, filed on Mar. 4, 2009.

(51) Int. Cl. A61F 2/04

(52) U.S. Cl. 600/37

(57) ABSTRACT

This application relates to cardiac medical devices, and specifically to adjustable tensioning devices for tissue anchors, to the intraventricular cardiac anchoring or banding devices themselves, to devices and methods for controlling the depth of penetration of tissue anchors, to devices and methods for the joining of both papillary muscles to the mitral valve, to devices and methods for non-invasive “sling” or “loop” tethering of papillary muscles, to devices and methods for establishing suction prior to and during tissue anchor implant, to a double-barreled needle delivery device, and to a method of remodelling the heart muscle by implanting one or more tethers, and then periodically reducing the tether distance over time.

### CROSS REFERENCE TO RELATED APPLICATIONS

Not applicable.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### REFERENCE TO A SEQUENCE LISTING

Not applicable.

### BACKGROUND

1. Field of the Invention

This application relates to cardiac medical devices, and specifically to adjustable tensioning devices for tissue anchors, to the intraventricular cardiac anchoring or banding devices themselves, to devices and methods for controlling the depth of penetration of tissue anchors, to devices and methods for the triple joining of both papillary muscles to the mitral valve, to devices and methods for non-invasive “sling” or “loop” tethering of papillary muscles, to devices and methods for establishing suction prior to and during tissue anchor implant, to a double-barreled needle delivery device, and to a method of remodelling the heart muscle by implanting one or more tethers, and then periodically reducing the tether distance over time.

2. Background of the Invention

According to the Center for Disease Control, heart disease is the leading cause of death in the United States and is a major cause of disability. Almost 700,000 people die of heart disease in the U.S. each year. That is about 29% of all U.S. deaths. Heart disease is a term that includes several more specific heart conditions.

One of these conditions is cardiomyopathy. Cardiomyopathy is a weakening of the heart muscle or a change in heart muscle structure. It often results in inadequate heart pumping or other heart function abnormalities. These can result from various causes, including prior heart attacks, viral or bacterial infections, and others.

The geometry of the myocardium is critical to proper functioning. The myocardium is comprised of a single, continuous tissue that wraps around itself, spiraling up from the apex of the heart, to form a helix with elliptically shaped ventricles. This spiral produces an oblique muscle fiber orientation, meaning that the fibers form a more ventricle ‘x’ shape, so that when fibers shorten 15%, it produces a 60% ejection fraction. Because of its elliptical shape and defined apex, the ventricle is subjected to a relatively low level of lateral stress.

However, a dilated left ventricle is generally due to the effects of a myocardial infarction or various disease state not fully understood (cardiomyopathy or idiopathic cardiomyopathy). An occlusion, or blockage, of cardiac arteries results in either an akinetic (non-beating) or dyskinetic (irregular beating) tissue downstream from the occlusion. This downstream ventricular tissue is damaged, but since the volume of blood that fills the ventricle does not change, the damaged organ has to work harder to eject the blood. This increased load causes an increase in the radius of the ventricle and the thickness of the ventricular wall changes. Further, the shape of the heart, more specifically the left ventricle, becomes circular, the remaining myocardial tissue suffers from pathological hypertrophy, and the valve opening widens. As the ventricle dilates, the muscle fiber orientation, which is critical to a good ejection fraction, becomes transverse, or more horizontal. Subsequently, the ejection fraction decreases; a 15% shortening of muscle fibers now produces only a 30% ejection fraction. The lateral stress on the ventricle increases. Overall, the dilated left ventricle cannot produce a strong enough pulse to maintain health and efficient circulatory return.

Ventricular reduction is a well-known type of operation in cardiac surgery to reduce enlargement of the heart from cardiomyopathy. In 1985, Vincent Dor, MD, introduced endoventricular circular patch plasty (EVCPP), or the Dor procedure, as a viable method for restoring a dilated left ventricle to its normal, elliptical geometry. The Dor procedure, which uses a circular suture and a Dacron® patch to correct LV aneurysms and exclude scarred parts of the septum and ventricular wall, has been one option for ventricular remodeling. Some have observed that the procedure restores ventricular shape, increases ejection fraction, decreases the left ventricular end systolic volume index (LVESVI), and allows for complete coronary revascularization.

The disadvantage to the Dor procedure is that it places synthetic tissue inside the LV cavity and it is usually done as part of a coronary artery bypass graft (open heart) surgery.

Others have attempted further solutions to this problem. U.S. Pat. No. 7,060,021 to Wilk discloses a type clamp for the left ventricle which pulls opposing walls of the heart together in order to close off lower portions of both ventricles.

U.S. published patent application 2007/0083076 to Lichtenstein discloses methods and devices for altering the blood flow through the left ventricle by engaging the outer surface of the heart in a type of binding.

U.S. published patent application 2008/0293996 to Evans discloses a system and method for volume reduction by inserting a conical polymeric container, i.e. balloon, into the left ventricle to reduce the volume of blood flow.

Additionally, many patents and publications are directed to the catheter based repair of the mitral valve using various types of sutures and tethers. For example, U.S. published patent application 2008/0243150 to Starksen discloses a valve annulus treatment device secured by anchors that cinch or draw together circumferentially to tighten the valve annulus (ring). Starksen also discloses that such a device can be delivered by advancing a catheter through the aorta. Published PCT patent application WO/2006/135536 to De Marchena discloses a papillary muscle tether for left ventricular reduction by delivery either (1) through the femoral vein and delivered to the left ventricle via a trans-septal approach into the left atrium, across the mitral valve, or (2) retrograde through the femoral artery, advanced through the aortic valve, and into the left ventricle. However, cardiac catheterization poses the risk of blood clots that can trigger strokes, damage to blood vessels, and damage to the heart or pericardium. Thus, procedures and devices which address these and other concerns are needed in the field.

Advances have been made in techniques and tools for use in minimally invasive surgery that can be performed through small incisions or intravascularly. For example, improvements have been made recently to reduce the invasiveness of cardiac surgery. To avoid open procedures, such as open, on pump, stopped-heart surgery, which can lead to high patient morbidity and mortality, devices and methods have been developed for operating through small incision, for operating on a beating heart, and for performing cardiac procedures via intravascular or intravascular access. Recently surgeons have begun performing procedures within a beating heart by operating on the heart via a small incision in the apex of the heart or other access point. For many minimally invasive surgery techniques, significant challenges include positioning the treatment device or devices in a desired location for performing the procedure and deploying the treatment into or on the target tissue.

Heart valve repair can benefit from less invasive surgical techniques. Traditional treatment of heart valve stenosis or regurgitation, such as mitral or tricuspic regurgitation, typically involves an open-heart surgical procedure to replace or repair the valve. Valve repair procedures usually involve annuloplasty, which is a set of techniques designed to restore the valve annulus shape and strengthen the annulus. Conventional annuloplasty surgery generally requires a thoracotomy (a large incision into a patient's thorax), and sometimes a median stemotomy (an incision through a patient's sternum). These open-heart, open-chest procedures routinely involve placing the patient on a heart- lung bypass machine for long periods of time so that the patient's heart and lungs can be stopped during the procedure. In addition, valve repair and replacement is typically technically challenging and requires a substantial incision through a heart wall to access the valve. Many patients such as elderly patients, children, patients with complicating conditions such as comorbid medical conditions or those having undergone other surgical procedures, and patients with heart failure, are not considered candidates for heart valve surgery because of the high risk involved.

Minimally invasive procedures are typically performed endoscopically through catheters, through small incisions or intravascularly or through external access to the heart through the apex, via an anterior lateral thorocotomy, or by approaching the heart laterally by making a small incision somewhere between the 4^{th} and 6^{th} inter costal ribs, exposing the beating heart. Instruments such as graspers, dissectors, clip appliers, lasers, cauterization devices and clamps are routinely used endoscopically, with an endo-scope used for visualizing the procedure. When a surgeon desires to bring two pieces of tissue together, the surgeon typically threads a suture through the two pieces of tissue, applies tension, and ties off or knots the suture to maintain the tension. However, during endoscopic surgery, the manipulation required when knotting or tying suture material can be difficult because of severely restricted space.

Previously, there have been attempts to maintain tension in tissue by using staples, clips, clamps, or other fasteners to obviate the need for suturing. However, these methods do not provide adjustable tension such as is available when a surgeon uses suture. U.S. Pat. Nos. 5,520,702 and 5,643,289 describe deformable cylindrical tubes that can be applied over a loop of suture. After a suture is adjusted to a desired tension, the suture is looped, and a deployment gun applies a deformable tube over the suture loop and crimps it so that it clamps down on the suture. After the loop is secured with a crimp, a separate cutting member or tool can be used to cut the excess suture material. U.S. Pat. No. 6,099,553 also describes deformable crimps that can be applied over the ends of sutures to fix them into place. Similar crimping devices that operate to mechanically fasten suture together and cut away excess tether are provided as TI-KNOT® knot replacement systems by LSI Solutions.® However, with crimping schemes, the suture may still slip through crimps and lose tension, especially if the suture has a small diameter, if the suture is made of a material susceptible to slippage, such as metal or TEFLON® fluoropolymer, or if the crimp is insufficiently deformed. U.S. Publication No. 2003/0167071 describes fasteners made from shape memory materials that can be applied to sutures to avoid tying knots in catheter-based procedures. U.S. Pat. Nos. 6,409,743 and 6,423,088 describe fusible collars that can be used in place of knots in securing sutures. These fusible collars require an external source of energy be locally applied to the collar without damaging surrounding tissue for the fusing process.

Devices and methods for less-invasive repair of cardiac valves have been described. In heart valve repair procedures, it is often desired for a physician to secure one or more treatment devices to valve annulus tissue. Annular tissue tends to be more fibrous than muscular or valve leaflet tissue, and thus can be more suitable tissue for securing treatment devices such as anchors to treat a heart valve or structural defect within the heart. Devices and methods for positioning anchor delivery devices are described in U.S. patent application Ser. Nos. 60/445,890, 60/459,735, 60/462,502, 60/524,922, 10/461,043, 10/656,797, 10/741,130 and 10/792,681, which were previously incorporated by reference. For example, these references describe devices and methods for exposing, stabilizing and/or performing a procedure on a heart valve annulus.

Many treatments, including annuloplasty, involve tightening of tissue. For some tissue tightening procedures, anchors coupled to a suture are embedded in tissue, and the suture is then cinched to tighten the tissue via the anchors. Examples of devices and methods for such procedures applied to heart valve repair are provided in U.S. patent application Ser. Nos. 10/656,797, 10/741,130 and 10/792,681.

Improved methods and devices for treating cardiomyopathy are needed.

### BRIEF SUMMARY OF THE INVENTION

In preferred embodiments, this application discloses devices and methods for adjustably tensioning tissue anchors, intraventricular cardiac anchoring or banding devices, devices and methods for controlling the depth of penetration of tissue anchors, devices and methods for the joining of both papillary muscles to the mitral valve, devices and methods for non-invasive “sling” or “loop” tethering of papillary muscles, devices and methods for establishing suction prior to and during tissue anchor implant, a double-barreled needle delivery device, and a method of remodelling the heart muscle by implanting one or more tethers, and then periodically reducing the tether distance over time.

In one preferred embodiment, the invention contemplates devices for easily adjusting the length of tissue tethers, methods of doing the same, specific tissue anchor assemblies, and methods of deploying and removing the same.

In a preferred embodiment, there is provided an adjustable in vivo tensioning actuator for connecting at least two implantable tissue tethers, comprising: a housing having one or more apertures capable of accepting an implantable tissue tether, and an adjustment mechanism that attaches to each implantable tissue tether during an in vivo endoscopic procedure and secures the tether to define the length of the tether and thereby the distance between the tissue anchors, wherein the tensioning actuator is reversibly adjustable without need for reinstallation of the tether implant.

In other preferred embodiments, there is provided an adjustable in vivo tensioning actuator as disclosed, further comprising wherein the adjustment mechanism comprises an axially disposed screw within said housing, wherein said implantable tissue tether winds about said axially disposed screw and said screw operates as a rotary shaft, or, further comprising wherein the adjustment mechanism comprises a common spring-loaded spooling mechanism attached to the housing by a lateral axle, wherein the implantable tissue tethers are fed into the housing via separate opposing apertures and the implantable tissue tethers wind about the common spring-loaded spooling mechanism, or, further comprising wherein the adjustment mechanism comprises a reversible clamp, or further comprising wherein there are three or more implantable tissue tethers, each of said tethers attached to a tissue anchor.

In another preferred embodiment, there is provided an adjustable tether implant, comprising: at least two elongated pieces of a medically appropriate implantable material for mechanically connecting two or more tissues together at a predefined distance, at least one tissue anchor attached to each of said elongated pieces, and the adjustable tensioning actuator described herein connected to the at least two elongated pieces for adjusting the distance between the tissue anchors, wherein the tensioning actuator is reversibly adjustable without need for reinstallation of the tether implant.

In another preferred embodiment, there is provided an adjustable tether implant as above, further comprising wherein the adjustment mechanism comprises an axially disposed screw within said housing, wherein said implantable tissue tether winds about said axially disposed screw and said screw operates as a rotary shaft.

In other preferred embodiments, there is provided an adjustable tether implant as above, further comprising wherein the adjustment mechanism comprises a common spring-loaded spooling mechanism attached to the housing by a lateral axle, wherein the implantable tissue tethers are fed into the housing via separate opposing apertures and the implantable tissue tethers wind about the common spring-loaded spooling mechanism, or, further comprising wherein the adjustment mechanism comprises a reversible clamp, or, further comprising wherein there are three or more implantable tissue tethers, each of said tethers attached to a tissue anchor.

In another preferred embodiment, there is provided a tissue anchor for implantable tethers, comprising: an anchor housing having a tip end, a wire-access end, and a tether fastening means, said housing defining an external shell and an inner cavity, at least one channel within said inner cavity, said at least one channel having a release wire partially disposed therein, said release wire having at one terminus a release loop located outside the anchor housing, and at the other release wire terminus the release wire is attached to a slidable filament core within said inner cavity, said slidable filament core having one or more filaments moveably attached thereto and extending from said inner cavity to outside the anchor housing for mechanical interaction with the tissue such that when the one or more moveable filaments are in a fully-extended open position they operate to secure the anchor to the tissue and when the one or more moveable filaments are in a folded closed position they do not securely engage the tissue, the slidable filament core having a disengagable locking mechanism such that when the release wire is pulled away from the anchor housing the slidable filament core slides within said inner cavity and the moveable filaments retract to a folded closed position enabling removal of the tissue anchor.

In other preferred embodiments, there is provided the tissue anchor above, further comprising wherein the moveable filaments have a tapered shape, or further comprising wherein the slidable filament core is attached to a spring which is mounted within the tip of the inner cavity, further comprising wherein the inner cavity comprises two channels or three or four channels, each channel having a branch wire from a split release wire comprised of a single external release wire at the release loop terminus and two internal branches of the release wire attached to the slidable filament core, or further comprising wherein the slidable filament core has a limiting mechanism wherein the filaments cannot rotate away from the tissue anchor housing beyond 90 degrees from the folded closed position when the tissue anchor is in the secure fully-extended open position.

In another preferred embodiment, there is provided a method for placing an anchor device within tissue, comprising the step of: inserting the tissue anchor described above into the target tissue wherein the tissue anchor is in a deployed position such that the tissue engaging filaments extend from the anchor housing in a manner to secure the tissue anchor to the tissue.

In another preferred embodiment, there is provided a method for standardizing the depth of penetration of the anchor device wherein a rim is placed around the body of the anchor behind the tissue engaging filaments to prohibit the anchor from penetrating the tissue beyond a preset depth.

In another preferred embodiment, there is provided a method of removing an anchor device from within tissue, comprising the steps of: pulling the release wire of the tissue anchor described herein to retract the tissue engaging filaments from the tissue and removing the anchor from said tissue.

In another preferred embodiment, a sling or band made of tether or other medically appropriate material is provided, to be threaded behind the papillary muscle for the tethering of such muscles, either to one another or to other tissue within the ventricle without requiring implantation anchors or other elements requiring the piercing of muscle tissue.

In another preferred embodiment, a method for engaging a sling or band made of tether or other medically appropriate material around the papillary muscle is provided, wherein the device is threaded between the papillary muscle and the ventricle wall to create a loop, which loop is then cinched in front of the muscle using a clamp or similar crimping device, allowing it to interact with the adjustable tensioning device described herein.

In another preferred embodiment, an anchoring system is provided that allows for total approximation of the opposing anchors, but which the anchors are drawn together with a filament or tether until they approximate each other, with the tether length minimized so as to bring the anchors as close together as possible, or the anchors are brought together in such a way as they interlock, with a “carabineer” like locking mechanism or a hook. This will have the effect of assuring that the opposing tissue is in effect joined with the opposite tissue—ensuring that opposing tissue moves together. For example, by totally approximating the papillary muscles if one of the papillary muscles is damaged or ischemic, improved coronary performance is assured by totally aligning the papillary muscles, from the tip to the base of each papillary muscle. This has the net effect of reducing the tenting of the chordea tendinea on the mitral valve and reducing mitral regurgitation and improving forward cardiac output.

### BRIEF DESCRIPTION OF THE DRAWINGS

FIG. 1A and 1B are line drawings representative of alternative embodiments of the tissue anchor.

FIG. 2 shows another mechanism for extending the filament arms, as shown from U.S. Pat. No. 4,637,757 for pilings technology.

FIG. 3 and FIG. 4 show various mechanisms for a buckle mechanism, as shown from patents in the mechanical strap technology.

FIG. 5 is a graphical representation of another embodiment showing a tether clamp mechanism having gripping teether and closure knobs/tracks.

FIG. 6 is a graphical representation of the locking clamp from a side view.

FIG. 7 is a graphical representation showing detail of the clamp mechanism in relation to the tissue tethers.

FIG. 8 is a graphical representation showing detail of a banding mechanism encircling a papillary muscle.

FIG. 9 is a graphical representation show detail of alternative anchors.

### DETAILED DESCRIPTION OF THE INVENTION

### Definitions

The following definitions are provided as an aid to understanding the detailed description of the present invention.

“Adjustable tether” or “adjustable tensioning device” is defined herein to mean the combination of an adjustable mechanism with an elongated piece of medically appropriate material for mechanically connecting two or more tissues together at a predefined distance.

The term “adjustable” refers to a mechanism which is accessible to a medical practitioner and operates to reversibly adjust the length of the tether within a few steps or less.

“Anchors” for the purposes of this application, is defined to mean any fastener. Thus, anchors may comprise C-shaped or semicircular hooks, curved hooks of other shapes, straight hooks, barbed hooks, clips of any kind, T-tags, or any other suitable fastener(s). In one embodiment, anchors may comprise two tips that curve in opposite directions upon deployment, forming two intersecting semi-circles, circles, ovals, helices or the like. In some embodiments, anchors are self-deforming. By “self-deforming” it is meant that anchors change from a first undeployed shape to a second deployed shape upon release of anchors from restraint in housing. Such self-deforming anchors may change shape as they are released from housing and enter papillary, myocardial or other muscle tissue, to secure themselves to the tissue. Thus, a crimping device or other similar mechanism is not required on distal end to apply force to anchors to attach them to tissue.

Self-deforming anchors may be made of any suitable material, such as a super-elastic or shape-memory material like Nitinol or spring stainless steel. In other embodiments, anchors may be made of a non-shape-memory material and made be loaded into housing in such a way that they change shape upon release. Alternatively, anchors that are not self-deforming may be used, and such anchors may be secured to tissue via crimping, firing or the like. Even self-securing anchors may be crimped in some embodiments, to provide enhanced attachment to tissue. In some embodiments, anchors may comprise one or more bioactive agent. In another embodiment, anchors may comprise electrodes. Such electrodes, for example, may sense various parameters, such as but not limited to impedance, temperature and electrical signals. In other embodiments, such electrodes may be used to supply energy to tissue at ablation or sub-ablation amounts. Delivery of anchors may be accomplished by any suitable device and technique, such as by simply releasing the anchors. Any number, size and shape of anchors may be included in housing.

Canula or cannula refers to a well-known tube-like medical instrument. It can be fitted with a trocar, a sharp pointed device for piercing tissue.

Tether may be one long piece of material or two or more pieces and may comprise any suitable material, such as Nitinol, austinetic steel, suture, suture-like material, a Dacron strip, GORE-TEX™ or the like.

Hemostasis valve, or valve/sleeve, refers to a device which allows the heart tissue to be pierced at the apex region with little or no blood loss. Similar valves/sleeves are well known in the venipuncture field where individual vacutainers can be repeatedly mounted on a single needle, and valves such as the Touehy Borst valve which allows multiple insertions of catheters while maintaining hemostasis.

General Method

The present application discloses methods and devices for reversibly adjusting an anchored tether. These methods generally involve securing to the tissue a first anchor that is coupled to an adjustable tether, securing to the tissue a second anchor that is slidably coupled to the tether, applying tension to the tether using the adjustment mechanism, and fixing the position of the tether with respect to the second anchor. Any or all of these steps can be performed intravascularly or through an incision in the beating heart. For example, tension can be applied to the tether intravascularly, and the anchors can be secured to the tissue intravascularly, or tension can be applied to the tether via an incision in the apex of the heart and the anchors can be secured through the apex of the heart.

Other Tissues

Although for exemplary purposes the following description typically focuses on uses of the disclosed methods and devices in mitral valve and other heart valve repair, such description should not be interpreted to limit the scope of the invention as defined by the claims. Tissue tightened by the disclosed methods and devices may comprise any part of the body including, for example, the heart, bladder, stomach, gastroesophageal junction, vasculature, gall bladder, or the like. The methods and devices disclosed herein may be used, for example, to close or reduce the diameter of any suitable body lumen, valve or structure or to tether portions of tissue which are separate or which have been traumatically severed.

Delivery

Generally, delivery of the tether device may be advanced by any suitable advancing or device placement method so long as it arrives at the location that the adjustable tether is required. Many catheter-based, minimally invasive devices and methods for performing intravascular procedures, for example, are well known, and any such devices and methods, as well as any other devices or method later developed, may be used to advance or position delivery device into a desired location.

For example, in one cardiac embodiment a steerable guide catheter is first advanced percutaneously to the cardiac region that the implanted adjustable tether will be anchored or otherwise attached. The steerable catheter is inserted into the region to be repaired, e.g. a ventricle of the heart, and thus into the space formed by left ventricle. An obturator pushes or holds the tissue in place once it has been pierced. Once in this space, the steerable catheter is easily advanced to the tissue to be repaired, for instance to the papillary muscle or to the ventricular wall. The anchor, band, sling or other tethering device may then be advanced and inserted into or otherwise attached to the papillary muscle, trabecula surrounding the papillary muscle, ventricular wall, mitral valve tissue, tissue of the anulus fibrosis sinister cordis, and/or the LV myocardium. Of course, this is but one exemplary method and any other suitable method, combination of devices, etc. may be used.

Antegrade and Retrograde

These devices can be placed using an antegrade endoscopic, or transvascular access technique for accessing the left ventricle from the right atrium, through transeptal puncture, through the left atrium, and down into the left ventricle. Alternatively, a retrograde technique, crossing the aorta and thereby into the left ventricle, is also contemplated as within the scope of the delivery method for the present invention.

Apical

These devices can be placed using an apical access technique for accessing the left ventricle through a small incision made in the apex of the beating heart. This permits easy access into a beating heart.

Placement of the invention is contemplated as being performed under ultrasound examination in order to optimize cardiac function during the installation procedure.

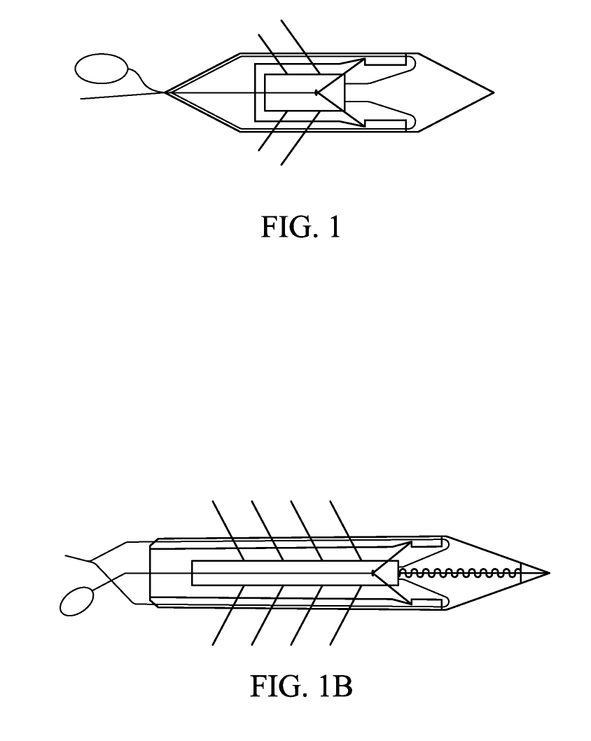

Referring now to the figures, FIGS. 1A and 1B are graphical line drawings representative of alternative embodiments of the tissue anchor. In FIG. 1A, details are presented with lead lines. In FIG. 1B, an embodiment having a spring within the tip of the housing for assisting implant and removal.

Specific Anchor Technology

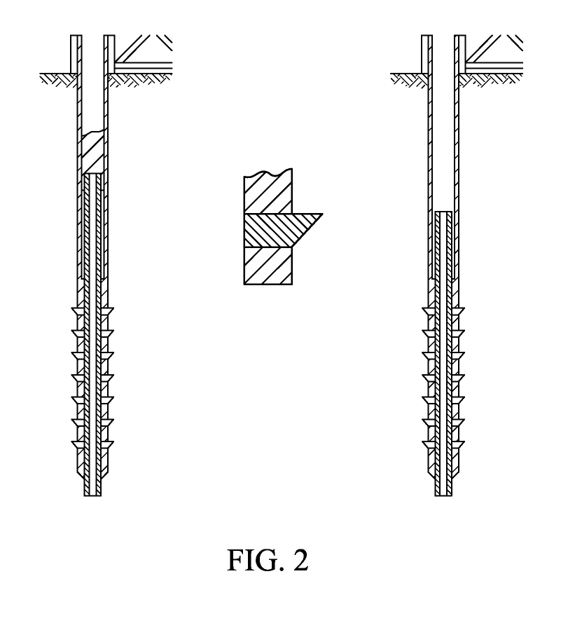

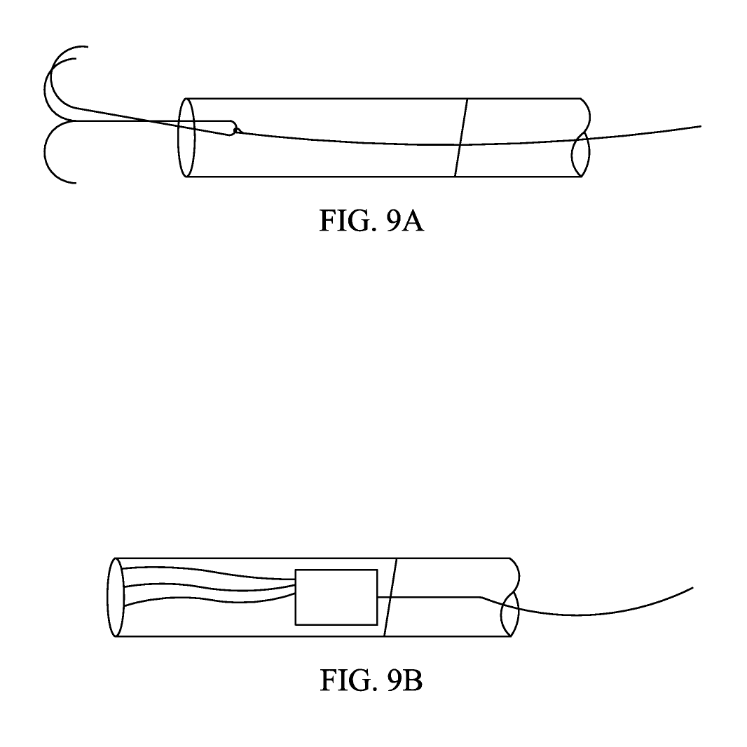

FIG. 2 shows another mechanism for extending the filament arms, as shown from U.S. Pat. No. 4,637,757 for seafloor pilings technology. Thus, the slidable filament core can drive the filaments into the tissue by way of a variety of mechanisms. FIG. 9 shows another embodiment of anchor technology.

Penetration Control

In one preferred embodiment, the anchor is deployed using a spring-loaded mechanism to speed up the time involved for the implant procedure. It is contemplated that implant of the device takes about 30 minutes or less. Another aspect of the spring-loaded feature is the ability to control depth of anchor insertion (3-8 mm), using a rim or other attachment to the device which prohibits the device from implanting beyond the preset depth. Anchors that are too shallow or too deep do not optimize attachment, as a 3-8 mm anchor would.

Adjustment Mechanisms

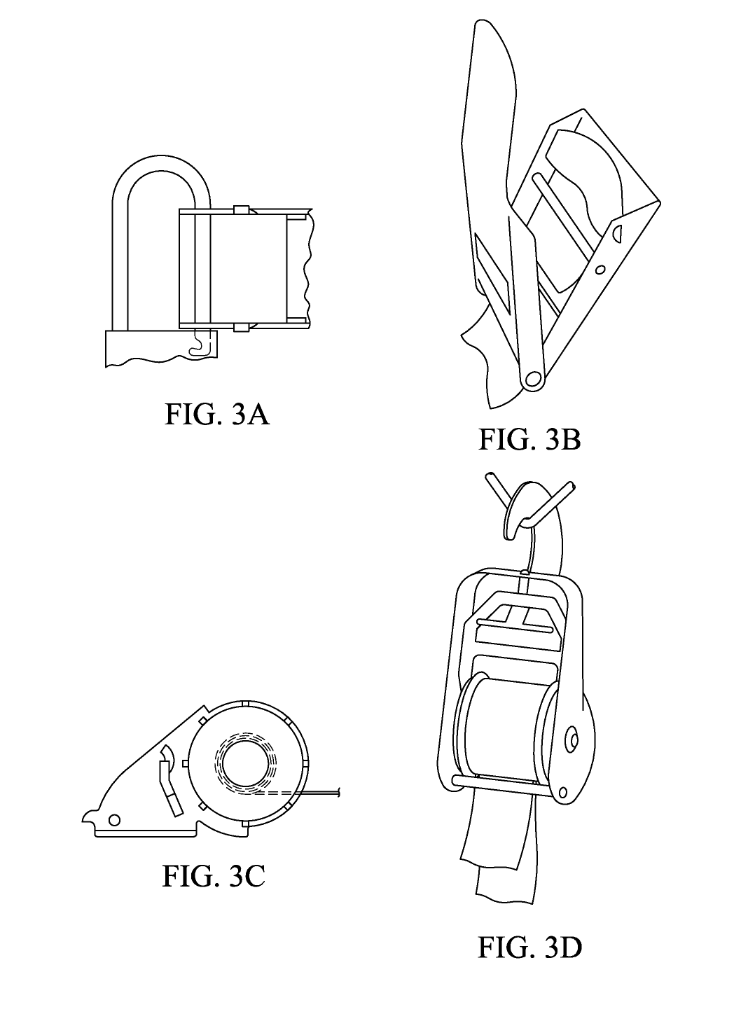

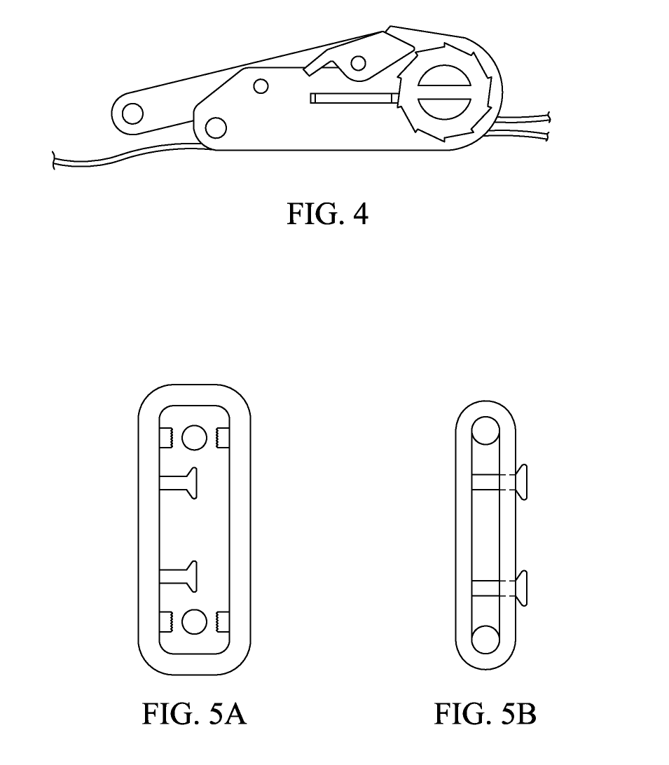

FIG. 3 and FIG. 4 show various buckle mechanisms, as shown from patents in the mechanical strap technology. Although not in the cardiac or surgical arts, it is within technical ability to manufacture appropriately sized versions of these or other buckles from medically appropriate materials. The important feature is the ability to tighten or loosen as becomes necessary depending on a patient's condition.

For the ratchet style buckle, it is contemplated that this mechanism can be driven by microelectromechanical devices for remotely controlling the lengthening or shortening of the tether without subsequent endoscopic intervention. Medical personnel could alter the left ventricular geometry while watching cardiac function in a patient while undergoing ultrasound examination.

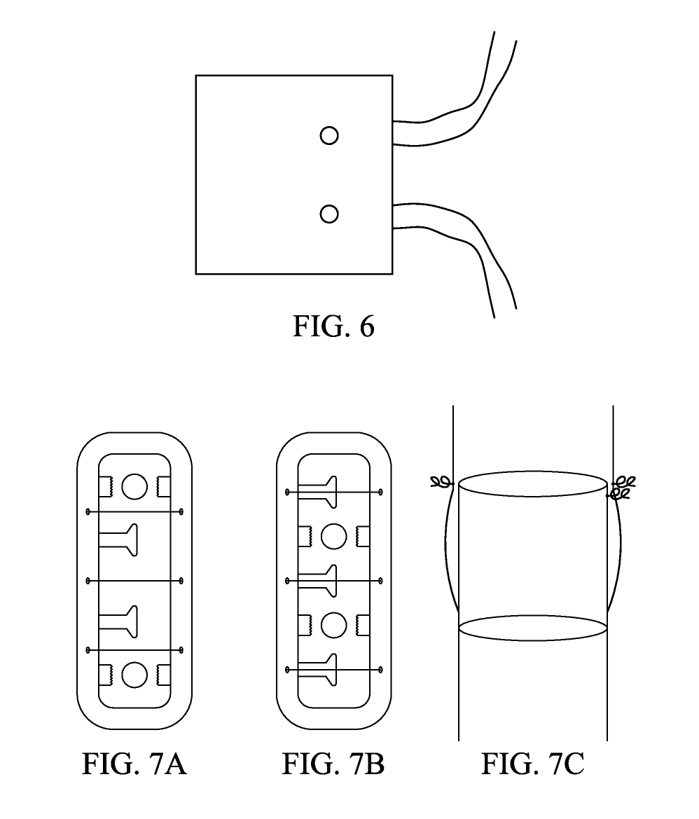

FIG. 5 is a graphical representation of another embodiment showing a tether clamp mechanism having gripping teether and closure knobs/tracks. FIG. 6 is a graphical representation of the locking clamp from a side view.

FIG. 7 is a graphical representation showing detail of the clamp mechanism in relation to the tissue tethers.

Papillary Alignment

In another preferred embodiment, the tether and anchor combination may be used for complete, 100% joining of posterior and anterior papillary muscles within the left ventricle. In contrast to the left ventricular adjustments described herein, the papillary muscle joinder provides another optional technique for cardiac care-givers provided by the secure anchoring of the removable anchors or bands and the adjustable nature of the tether system provided herein.

Papillary/Wall Alignment

In another preferred embodiment, the papillary muscle on one side of the left ventricle is attached, using the tether and anchors, to the wall tissue on the opposite side of the ventricle in lieu of the opposing papillary muscle. In contrast to the other left ventricular adjustments described herein, the papillary/wall muscle joinder provides another optional technique for cardiac care-givers provided by the secure anchoring of the removable anchors and the adjustable nature of the tether system provided herein.

Use of Non-Puncturing Band/Loop

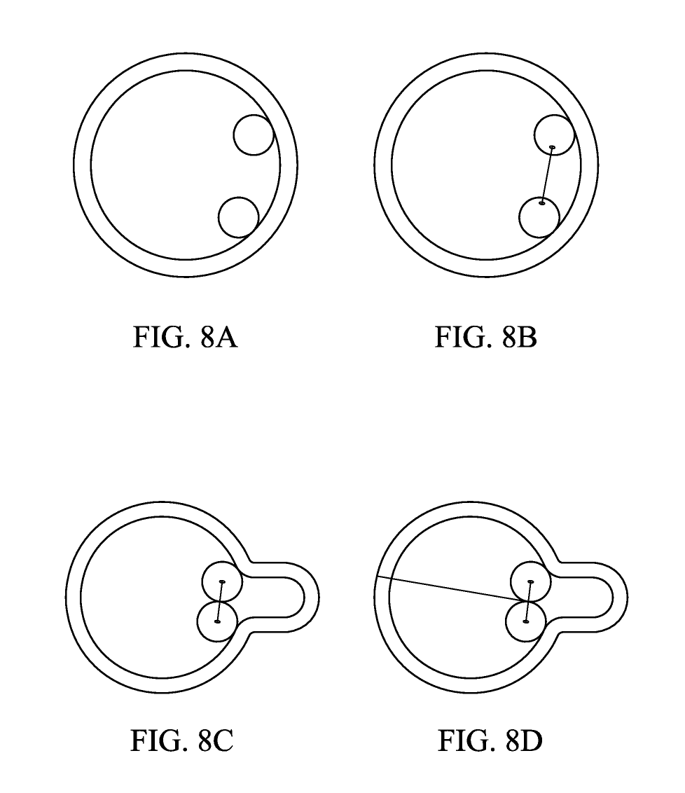

Referring to FIG. 8, in another preferred embodiment, using a delivery method identical or similar to the one described herein, the tether or another medically appropriate material is threaded through the space between the papillary muscle and the ventricular wall to which that muscle is attached, thereby creating a loop around the papillary muscle. The loop is then cinched in front of the muscle using a clamp or similar device, allowing the loop to interact with the adjustable tensioning device described herein. In this embodiment, it is contemplated to use a band, clip, suture, hook or anchor that is woven, installed, and pushed between the papillary muscles and trabaculation near the papillary muscles for the purposes of bringing the papillary muscles together as a way of joining the papillary muscles without puncturing the tissue.

Periodic Tensioning Method

In another preferred embodiment, one or more tethers are implanted to effectuate a remodeling of the cardiac muscle. The tethers may be intra-ventricular and may include tethers that connect papillary to papillary, papillary to septum, papillary to left ventricular wall, papillary to mitral valve, and combinations thereof. Upon implant, it is contemplated that the tether(s) will have little or no tension, thus giving the anchors a chance to “cure” into the cardiac muscle. Applying tension immediately after implanted can cause migration or dislodgment of the anchors. After approximately 30 to 45 days (however this may vary based on a doctor's observations), the tether is re-accessed, preferably using retrograde endoscopy but not limited to it. Once the tether is accessed, the tether is shortened 1 to 3 mm to establish tension and exert reduction pressure on the LV space. This procedure is then repeated, preferably every 3 months, where it may be shortened anywhere from 1 to 10 mm, over the course of a year or more. Remodeling of the cardiac muscle is thus effectuated and cardiomyopathy is treated.

Suction Feature

In another preferred embodiment, the use of suction is employed to verify muscle targeting and assist implanting of the tissue anchor. Using suction on the implant catheter allows the physician to distinguish between properly implantable tissue, e.g. muscle and, for example, undesirable tissue which might pose deleterious effects on the patient, e.g. chordae tendineae. Additionally, suction may be employed during implantation to ensure proper anchoring and penetration, and avoid attempts to anchor from an “air drop”, resulting in an improperly attached tissue anchor.

Triple Anchor to Treat Mitral Regurgitation

In another embodiment, the tethers are attached using tissue anchors to the posterior and the anterior papillary muscle, which are then drawn up towards the mitral valve by a third anchor. In is contemplated to attach anchors to join the papillary muscles—either base, mid or apex, preferably just the apex. Then, with a third anchor from the partially or totally joined papillary muscles, tether to the section of the top of the left ventricle between the aortic valve and the mitral valve. We then have an adjustor on that third tether that allows us to draw up the papillary muscles to the mitral valve. Allowing for enough of a reduction in tension to reduce/eliminate mitral regurgitation.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed:

1. An adjustable in vivo tensioning actuator for connecting at least two implantable tissue tethers, comprising: a housing having one or more apertures capable of accepting an implantable tissue tether or a tissue tether comprising a band or sling, and an adjustment mechanism that attaches to each tissue tether during an in vivo endoscopic procedure and secures the tether to define the length of the tether and thereby the distance between the tissue attachments, wherein the tensioning actuator is reversibly adjustable without need for reattachment of the tethers to the tissues being manipulated.

2. The adjustable in vivo tensioning actuator of claim 1, further comprising wherein the adjustment mechanism comprises an axially disposed screw within said housing, wherein said tissue tether winds about said axially disposed screw and said screw operates as a rotary shaft.

3. The adjustable in vivo tensioning actuator of claim 1, further comprising wherein the adjustment mechanism comprises a common spring-loaded spooling mechanism attached to the housing by a lateral axle, wherein the tissue tethers are fed into the housing via separate opposing apertures and the tissue tethers wind about the common spring-loaded spooling mechanism.

4. The adjustable in vivo tensioning actuator of claim 1, further comprising wherein the adjustment mechanism comprises a reversible clamp.

5. The adjustable in vivo tensioning actuator of claim 1, further comprising wherein there are three or more tissue tethers, each of said tethers attached to a tissue anchor.

6. An adjustable tether implant, comprising: at least two elongated pieces of a medically appropriate implantable material for mechanically connecting two or more tissues together at a predefined distance, at least one tissue anchor attached to each of said elongated pieces, and the adjustable tensioning actuator of claim 1 connected to the at least two elongated pieces for adjusting the distance between the tissue anchors, wherein the tensioning actuator is reversibly adjustable without need for reinstallation of the tether implant.

7. The adjustable tether implant of claim 6, further comprising wherein the adjustment mechanism comprises an axially disposed screw within said housing, wherein said implantable tissue tether winds about said axially disposed screw and said screw operates as a rotary shaft.

8. The adjustable tether implant of claim 6, further comprising wherein the adjustment mechanism comprises a common spring-loaded spooling mechanism attached to the housing by a lateral axle, wherein the implantable tissue tethers are fed into the housing via separate opposing apertures and the implantable tissue tethers wind about the common spring-loaded spooling mechanism.

9. The adjustable tether implant of claim 6, further comprising wherein the adjustment mechanism comprises a reversible clamp.

10. The adjustable tether implant of claim 6, further comprising wherein there are three or more implantable tissue tethers, each of said tethers attached to a tissue anchor.

11. A tissue anchor for implantable tethers, comprising: an anchor housing having a tip end, a wire-access end, and a tether fastening means, said housing defining an external shell and an inner cavity, at least one channel within said inner cavity, said at least one channel having a release wire partially disposed therein, said release wire having at one terminus a release loop located outside the anchor housing, and at the other release wire terminus the release wire is attached to a slidable filament core within said inner cavity, said slidable filament core having one or more filaments moveably attached thereto and extending from said inner cavity to outside the anchor housing for mechanical interaction with the tissue such that when the one or more moveable filaments are in a fully-extended open position they operate to secure the anchor to the tissue and when the one or more moveable filaments are in a folded closed position they do not securely engage the tissue, the slidable filament core having a disengagable locking mechanism such that when the release wire is pulled away from the anchor housing the slidable filament core slides within said inner cavity and the moveable filaments retract to a folded closed position enabling removal of the tissue anchor.

12. The tissue anchor of claim 11, further comprising wherein the moveable filaments have a tapered shape.

13. The tissue anchor of claim 11, further comprising wherein the slidable filament core is attached to a spring which is mounted within the tip of the inner cavity.

14. The tissue anchor of claim 11, further comprising wherein the inner cavity comprises two channels each channel having a branch wire from a split release wire comprised of a single external release wire at the release loop terminus and two internal branches of the release wire attached to the slidable filament core.

15. The tissue anchor of claim 11, further comprising wherein the slidable filament core has a limiting mechanism wherein the filaments cannot rotate away from the tissue anchor housing beyond 90 degrees from the folded closed position when the tissue anchor is in the secure fully-extended open position.

16. A method for placing an anchor device within tissue, comprising the step of: inserting the tissue anchor of claim 11 into the target tissue wherein the tissue anchor is in a deployed position such that the tissue engaging filaments extend from the anchor housing in a manner to secure the tissue anchor to the tissue.

17. A method for standardizing the depth of penetration of an anchor device within tissue, comprising the step of: providing a rim or collar device around the housing of the tissue anchor of claim 11, wherein the rim or collar is located behind the tissue engaging filaments to prohibit the anchor from penetrating the tissue beyond a preset depth.

18. A method of removing an anchor device from within tissue, comprising the steps of: pulling the release wire of the tissue anchor of claim 11 to retract the tissue engaging filaments from the tissue and removing the anchor from said tissue.

19. A method of tethering cardiac papillary muscles, comprising the step of: threading a sling or band made of medically implantable material around a papillary muscle, wherein said threading allows tethering of said papillary muscle, either to another papillary muscle or to other tissue within the ventricle, wherein said threading provides tethering without requiring implantation anchors or other elements requiring the piercing of muscle tissue.

20. The method of claim 19, further comprising the steps of: threading the sling or band of medically implantable material between the papillary muscle and the ventricle wall to create a loop, cinching said loop in front of the papillary muscle using a clamp or similar crimping device, and allowing it to interact with the adjustable tensioning device of claim 1.

21. An anchor system for allowing synchronous movement of tethered tissue, comprising: two or more medically implantable tissue anchors in combination, wherein said tissue anchors have one or more hook or drawstring elements attached thereby allowing each tissue anchor to become interjoined with at least one opposing tissue anchor, wherein said one or more hook or drawstring elements allow the tissue to completely join together, allowing the tissue to move in a synchronous manner.

22. The anchor system of claim 21, further comprising wherein the tethered tissue is papillary tissue within the left ventricle.

23. A medically implantable tissue anchor that forms a circle or semi circle upon implantation, wherein the tissue anchor possesses barbs that form a full 360 degree circle, wherein there is no part of the barb exposed to inside of the heart or tissue should a partial implant be the result of the implantation procedure.

24. An anchor system for reduction of mitral regurgitation, comprising: at least two tethers that are attached using tissue anchors to the posterior and the anterior papillary muscle, and a third anchor attached to the top of the left ventricle between the aortic valve and the mitral valve or across the left ventricle to the septum, and an adjustor on that third tether that allows the papillary muscle tethers to be drawn toward the mitral valve, wherein the reduction in papillary to mitral valve distance reduces/eliminates mitral regurgitation.

25. A method of implanting a tissue anchor using suction to verify target muscle and reduce damage to surrounding tissue, comprising: providing suction within the implantation canula of an endoscopy system during a cardiac tethering procedure, wherein the suction provides enhanced muscle contact with the implantation canula.

26. An adjustable tether implant, with a slidable core, adjustable whether by tensioning or loosing, that is implanted for 30-45 days prior to tightening to allow for the formation of adequate fibrous tissue, increasing the tensile strength of the anchor, that is adjustable in 1 mm increments via an adjustor mechanism on subsequent procedures for the purpose of tightening or loosening the tensile strength of the device, for the purpose of resisting diastolic pressure on the heart.

27. The adjustable tether implant of claim 26, further comprising wherein the slidable core has 2, 3 or four layers of slidable filament, so allow for maximum tightening that can be ratcheted, tightening or loosening the tensioning element or tendon via small, incremental rachets within the slidable filament.

28. The adjustable tether implant of claim 27, further comprising wherein one or more anchors can be released after implant or by abandoning the procedure, leaving the anchor permanently implanted in the heart.
